(19) United States
(12) Patent Application Publication
(10) Pub. No.: US 2014/0243766 A1
(43) Pub. Date: Aug. 28, 2014

(54) VACUUM ASSISTED HEALING PUMP FOR POST RADIATION AND CHEMOTHERAPY WOUNDS OF THE BREAST

(71) Applicant: Valerie Kay Martuch, Fenton, MI (US)

(72) Inventor: Valerie Kay Martuch, Fenton, MI (US)

(21) Appl. No.: 13/776,572

(22) Filed: Feb. 25, 2013

(51) Int. Cl. A61M 1/00

(52) U.S. Cl. CPC A61M 1/0031
USPC 604/319

(57) ABSTRACT

A vacuum assisted healing pump promotes healing of radiation and chemotherapy wounds of the breast. The vacuum assisted healing pump can increase the probability of healing, preventing the necessity of breast removal. The vacuum assisted healing pump applies localized vacuum to a wound, pulling blood into damaged blood vessels to promote healing and improve the viability of the surrounding tissue over time. The vacuum applied is from about 125 to about 300 mm of mercury, typically from about 225 to about 250 mm of mercury. The vacuum assisted healing pump has been used to reduce a wound, that hadn't healed for over two years, about 50% in size over a period of six weeks.

### BACKGROUND OF THE INVENTION

The present invention relates to wound healing methods and devices and, more particularly, to a vacuum assisted healing pump for post radiation and chemotherapy wounds of the breast.

Standard breast cancer treatment includes radiation. Radiation treatments damage the tissue and reduce its ability to heal. When post radiation wounds occur, from additional surgery or injury, the wounds do not heal well due to the damaged blood vessels. This can lead to an open, draining wound in the subject breast. Current medical protocol would typically lead to removal of the subject, damaged breast.

Conventional vacuum therapies are not used on the breast and are not intended for irradiated tissue of the breast. The conventional vacuum therapies use large machines for large, open wounds and such machines would not work well on smaller, open wounds on the breast.

Machines currently on the market require invasive application of large sponges and bandages under nurse or doctor care and application. This can require several office visits, increasing the time, cost and difficulty for the treatment.

As can be seen, there is a need for an improved method and apparatus for vacuum assisted healing of post radiation and chemotherapy wounds of the breast.

### SUMMARY OF THE INVENTION

In one aspect of the present invention, a vacuum assisted healing pump comprises a suction cone operable to be sealed over wound tissue; a vacuum pump providing vacuum inside the suction cone; a vacuum adjustment mechanism for adjusting vacuum pressure between about 125 mm mercury to about 300 mm mercury; and a fluid collection bottle disposed between the suction cone and the vacuum pump, the fluid collection bottle collecting and measuring bodily fluid removed from the wound tissue.

In another aspect of the present invention, a method for treating post-radiation wounds comprises sealing a suction cone over wound tissue; operating a vacuum pump to provide a vacuum inside the suction cone between about 125 mm mercury to about 300 mm mercury; and collecting and measuring bodily fluids from the wound tissue in a fluid collection bottle.

These and other features, aspects and advantages of the present invention will become better understood with reference to the following drawings, description and claims.

### BRIEF DESCRIPTION OF THE DRAWINGS

FIG. 1 is a schematic system diagram of a vacuum assisted healing pump according to an exemplary embodiment of the present invention;

FIG. 2 is a schematic functional diagram of the vacuum assisted healing pump of FIG. 1, in use with wound tissue of a user's breast;

FIG. 3 is a schematic functional diagram of a portable, battery-powered vacuum assisted healing pump, in use with wound tissue of a user's breast; and

FIG. 4 is a schematic functional diagram of a programmable vacuum-control vacuum assisted healing pump, in use with wound tissue of a user's breast.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a vacuum assisted healing pump that can promote healing of radiation and chemotherapy wounds of the breast. The vacuum assisted healing pump can increase the probability of healing, preventing the necessity of breast removal. The vacuum assisted healing pump applies localized vacuum to a wound, pulling blood into damaged blood vessels to promote healing and improve the viability of the surrounding tissue over time. The vacuum applied is from about 125 to about 300 mm of mercury, typically from about 225 to about 250 mm of mercury. The vacuum assisted healing pump has been used to reduce a wound, that hadn't healed for over two years, about 50% in size over a period of six weeks.

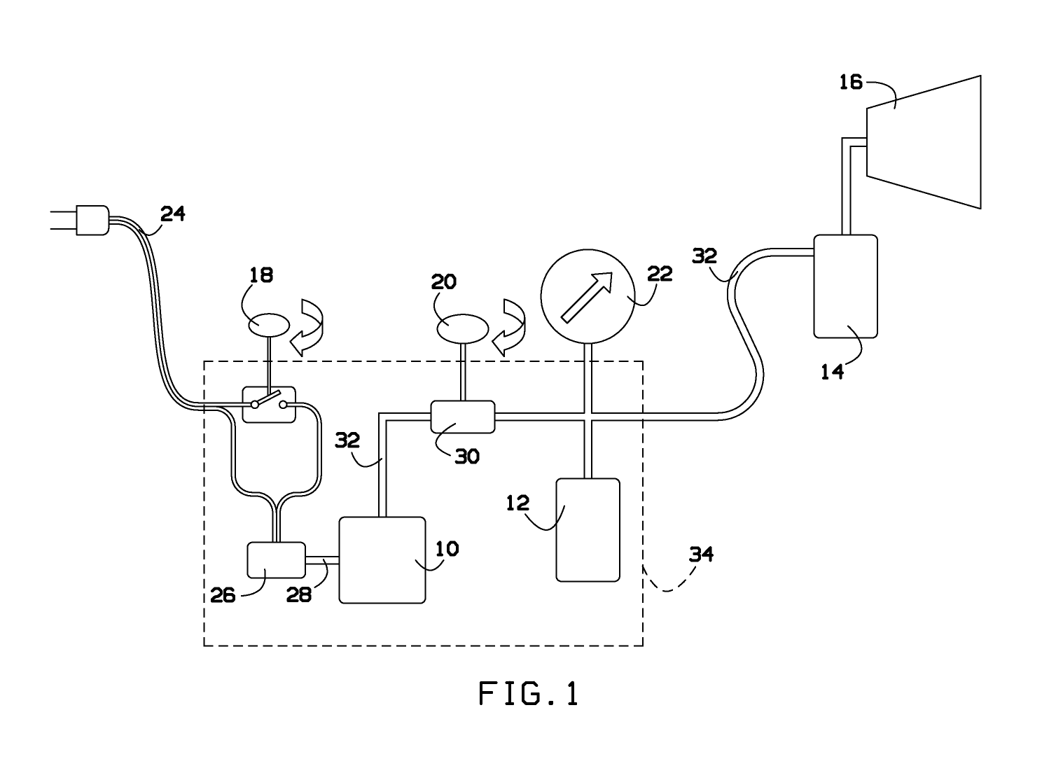

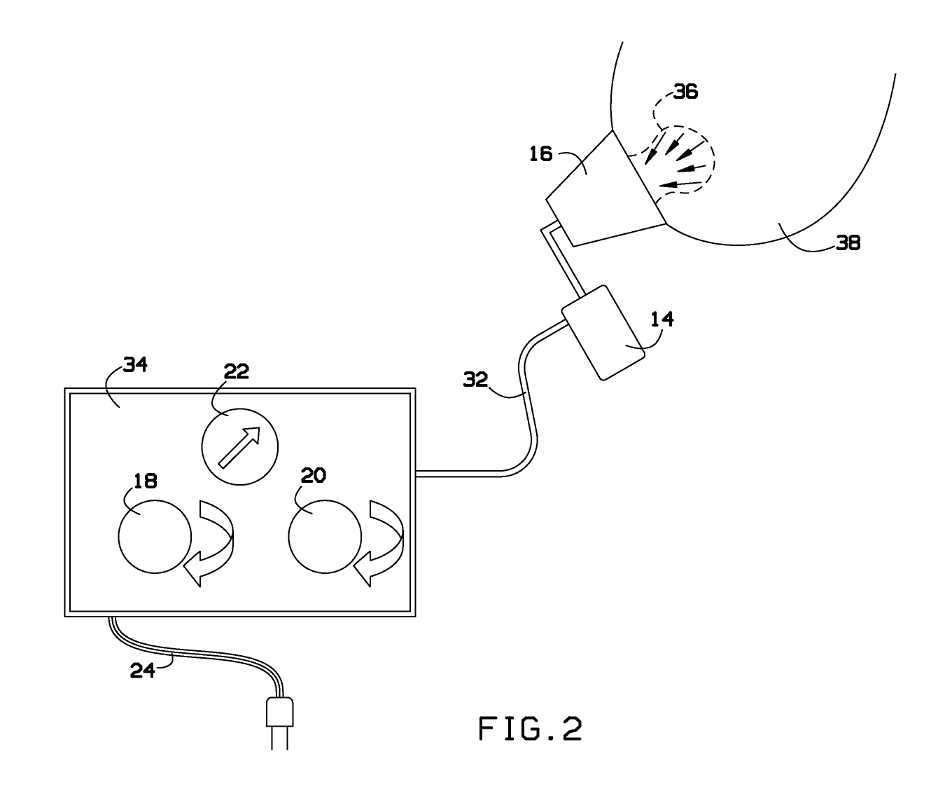

Referring to FIGS. 1 and 2, a vacuum unit 34 can be designed to provide an appropriate vacuum to a suction cone 16 that can be applied to wound tissue 36 of a breast 38. Typically, the vacuum unit 34 can provide up to about 300 mm mercury of vacuum. A user could apply the suction cone 16 to their wound tissue 36 a predetermined number of times for a predetermined period of time. For example, the user could apply the suction cone 16 to their wound tissue 36 from two to three times a day, for 10 to 30 minutes each treatment. The suction cone 16 can be removably attached to a vacuum hose 32 so that a user can change or clean the suction cone 16 as desired. Typically, the suction cone 16 is sized from about 5 mm to about 15 mm larger than the wound size. The suction cone 16 can include a soft, pliable ring about its opening to help form a seal over the wound tissue 36.

The vacuum unit 34 can include a vacuum pump 10 for drawing a vacuum. The vacuum pump 10 can be powered by a pump motor 26 connected to the vacuum pump 10 via a pump drive shaft 28. The pump motor 26 can be powered through an on/off switch 18. The on/off switch 18 can include an optional timer so that when the switch 18 is closed (and the vacuum unit 34 is turned on), the switch 18 remains closed for a predetermined period of time, such as from about 10 to about 30 minutes. An AC/DC power supply cord 24 can connect the vacuum unit 34 to a power supply. While the Figures show the pump motor 26 being separate from the vacuum pump 10, in some embodiments, the vacuum pump may include the pump motor integrated therewith as a single component.

Vacuum hose 32 can pass from the vacuum pump 10 to a vacuum control valve 30 having a vacuum adjustment knob 20. A user can turn the vacuum adjustment knob 20 to control the maximum amount of vacuum that can be realized in the suction cone 16. A vacuum gauge 22 can be used to provide a user with an accurate representation of the amount of vacuum generated. In some embodiments, the vacuum adjustment knob 20 can be omitted and the vacuum pump 10 can be designed and controlled to pull the appropriate vacuum without requiring user adjustment. In other embodiments, an internal vacuum adjustment can be provided, where a healthcare provider can adjust the specific vacuum delivered for each patient's needs.

A vacuum accumulator 12 can be provided in the vacuum unit 34 between the vacuum pump 10 and the suction cone 16. The vacuum accumulator 12 can be fluidly connected with the vacuum hose 32 to provide a volume to draw vacuum wherein. This volume can help reduce or eliminate small fluctuations in vacuum pressure due to pulses from the vacuum pump 10.

The vacuum hose 32 can extend from the vacuum unit 34 and connect to a fluid collection bottle 14 before terminating at the suction cone 16. The fluid collection bottle 14 can be used to collect any bodily fluids that might be collected. These fluids can be measured and discarded as may be recommended by the user's doctor or nurse. The fluid collection bottle 14 also prevents these fluids from collecting in the vacuum accumulator 12 or from passing into the vacuum pump 10. The fluid collection bottle 14 can be disposed at any location along the vacuum hose 32, typically close to the suction cone 16.

While the Figures show the suction cone 16 disposed separate from the vacuum unit 34 and connected thereto with the vacuum hose 32, in some embodiments, the suction cone 16 can be integrated with the vacuum unit 34 to provide a one-piece device that a user can apply vacuum to their wound.

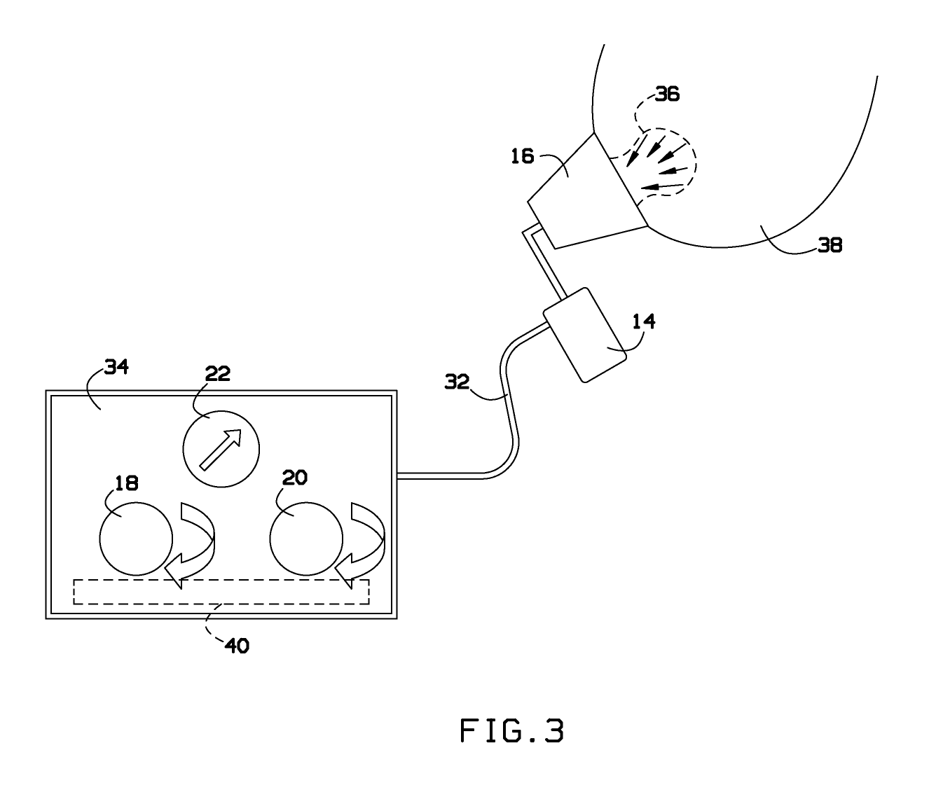

Referring now to FIG. 3, in some embodiments, the vacuum unit 34 can be powered by a rechargeable battery pack 40. The battery pack 40 can be internal to the vacuum unit 34 and an electrical connection (not shown) can be provided to connect power to the vacuum unit 34 to recharge the battery pack 40. In some embodiments, the battery pack 40 can be a plug-in battery pack that can be removed, exchanged and recharged. In some embodiments, a power cord (such as AC/DC power supply cord 24 from FIG. 1) can be provided along with the battery pack 40, providing a dual power source while keeping the battery pack 40 charged while the power supply cord is plugged in. The battery pack 40 can provide a portable device for use at times where other power supplies may not be readily available.

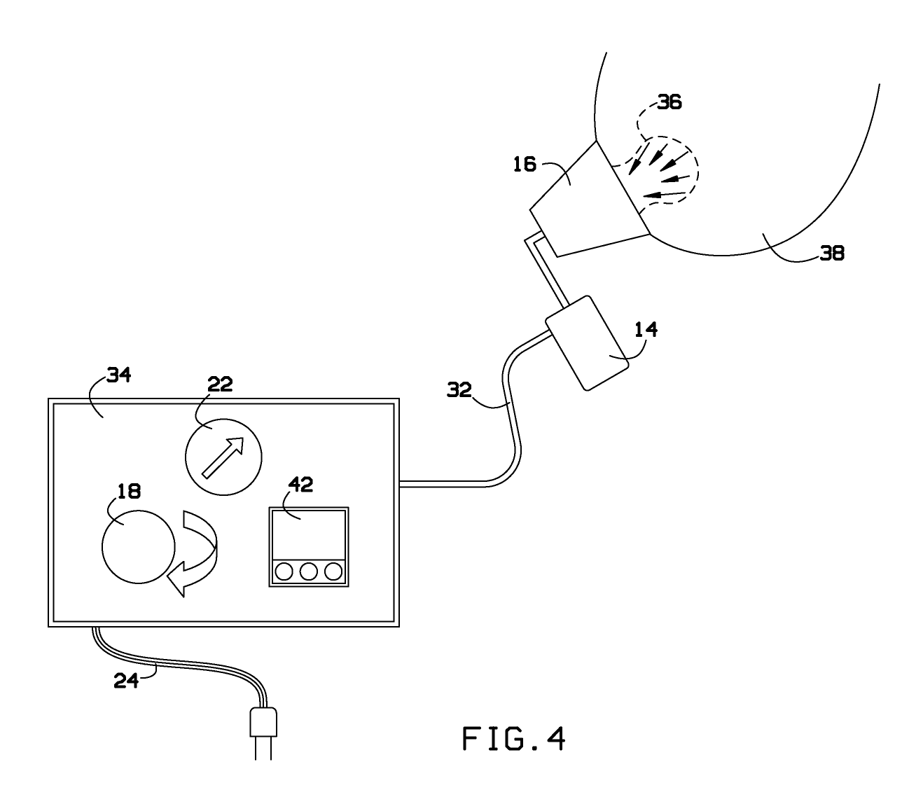

Referring to FIG. 4, a programmable vacuum control 42 can be provided on the vacuum unit 34. The programmable vacuum control 42 can include a display allowing the user to set the desired vacuum pressure provided by the vacuum pump 10. The programmable vacuum control 42 can include a lock-out feature so that a doctor or nurse can set the vacuum pressure and the end user would be prevented from changing the pressure. In some embodiments, the programmable vacuum control 42 can include a memory to track usage so that a healthcare provider can track the amount the device is used.

While the above description focuses on treatment of wounds of the breast, the device of the present invention can be used to promote healing of other wounds that do not heal due to radiation or diabetes. Diabetic ulcers of the legs and feet, if small enough, could be treated with the vacuum assisted healing pump of the present invention.

It should be understood, of course, that the foregoing relates to exemplary embodiments of the invention and that modifications may be made without departing from the spirit and scope of the invention as set forth in the following claims.

What is claimed is:

1. A vacuum assisted healing pump comprising:
a suction cone operable to be sealed over wound tissue;
a vacuum pump providing vacuum inside the suction cone;
a vacuum adjustment mechanism for adjusting vacuum pressure between about 125 mm mercury to about 300 mm mercury; and
a fluid collection bottle disposed between the suction cone and the vacuum pump, the fluid collection bottle collecting and measuring bodily fluid removed from the wound tissue.

2. The vacuum assisted healing pump of claim 1, further comprising a vacuum accumulator fluidly connected to a vacuum hose interconnecting the vacuum pump with the suction cone.

3. The vacuum assisted healing pump of claim 1, wherein the vacuum adjustment mechanism includes a vacuum adjustment knob connected with a vacuum control valve.

4. The vacuum assisted healing pump of claim 1, wherein the vacuum adjustment mechanism includes a programmable vacuum control.

5. The vacuum assisted healing pump of claim 1, further comprising a vacuum gauge measuring an amount of vacuum provided in the suction cone.

6. The vacuum assisted healing pump of claim 1, further comprising a rechargeable battery pack providing power to the vacuum pump.

7. The vacuum assisted healing pump of claim 1, further comprising an on/off switch for controlling power supplied to the vacuum pump.

8. The vacuum assisted healing pump of claim 7, wherein the on/off switch includes a timer.

9. A method for treating post-radiation wounds comprising:
sealing a suction cone over wound tissue;
operating a vacuum pump to provide a vacuum inside the suction cone between about 125 mm mercury to about 300 mm mercury; and
collecting and measuring bodily fluids from the wound tissue in a fluid collection bottle.

10. The method of claim 9, wherein the vacuum is provided over the wound tissue for a time period from about 10 minutes to about 30 minutes.

11. The method of claim 9, wherein the wound tissue is breast tissue.
